(19) United States
(12) Patent Application Publication
(10) Pub. No.: US 2022/0204423 A1
(43) Pub. Date: Jun. 30, 2022

(54) CASHEW NUT SHELL LIQUID BASED SUBSTITUTED CYCLOHEXENE DERIVATIVES AND USES THEREOF

(71) Applicant: CARDOLITE CORPORATION, Bristol, PA (US)

(72) Inventors: Joseph R. Mauck, Newwtown, PA (US); Anbazhagan Natesh, North Wales, PA (US); Chetan Tambe, Morganstown, WV (US); Pietro Campaner, Trieste (IT); Fernanda Tavares, Holland, PA (US); Timothy Stonis, Newtown, PA (US)

(21) Appl. No.: 17/394,745

(22) Filed: Aug. 5, 2021

Related U.S. Application Data
(60) Provisional application No. 63/064,473, filed on Aug. 12, 2020.

(51) Int. Cl. C07C 1/24; C07C 29/20; C07D 303/04; C07D 301/03; C07F 7/08; C07C 67/035

(52) U.S. Cl. CPC C07C 1/24; C07C 29/20; C07C 67/035; C07D 301/03; C07F 7/0834; C07D 303/04

(57) ABSTRACT

The present invention describes novel Cashew Nut Shell Liquid derived cycloaliphatic functional compounds and methods for making the same. The invention also provide methods to use these derivatives in antimicrobials, antioxidants, adhesives, coatings, corrosion retardants composites, cosmetics, detergents, soaps, de-icing products, elastomers, food, flavors, inks, lubricants, oil field chemicals, tackifiers, prepolymer chain-extenders, rheology modifiers, electrical and electronic components (potting, castings, encapsulants), personal care products, polymers, structural polymers, engineered plastics, 3D printable polymers, 3D printable polymers, UV/E-beam/cationic curable polymers, techno-polymers, rubbers, sealants, solvents, surfactants and varnishes, transformer oil, lubricants.

### BACKGROUND OF THE INVENTION

### Field of the Invention

Embodiments described herein generally relate to Cashew Nut Shell Liquid derivatives and methods for making and using the same. In particular, such embodiments relate to Cashew Nut Shell Liquid based cycloaliphatic compounds and their uses as such and as novel polymer building blocks for making a wide variety of products.

### Description of the Related Art

Bio-based derivatives represent valid alternatives to commonly used petro-based resources, thanks to their availability, sustainability and peculiar chemical structures. However, bio-based materials have showed some limitations relating to limited yields and selectivity, by-products formation, use of raw materials coming from the food chain, lack of batch-to-batch consistency and subsequent reproducibility, with an unavoidable impact on economic value and industrial scalability.

Cashew Nut Shell Liquid (CNSL) is a well-known non-edible natural oil obtained as a by-product of the Anacardium occidentale nut. CNSL is one of the most widely used bio-based resource to provide useful chemicals for coatings, adhesives, sealants and elastomers (CASE) applications. When distilling CNSL under proper temperature and pressure conditions, the main component of CNSL, anacardic acid, decarboxylates, allowing the recovery of cardanol. Even if cardanol represents an interesting and versatile molecule, thanks to its alkenyl-phenolic structure, there are applications and sectors where the use of cardanol is still quite limited due to lack of suitable building block derivatives. In fact, many cardanol derivatives reported in the literature are based on cardanol as mixture of isomers, with subsequent potential lack of reproducibility, limited average type of functional groups and applicability in sectors where color and purity are key aspects (e.g. 1K polyurethanes, thermoplastics). Moreover, many cardanol-derivatives reported in the literature and used at industrial level are still characterized by the presence of the aromatic ring. Even if its functionalization (through condensation, alkylation, alkoxylation of the phenoxy group) allow the recovery of a wide range of functional monomers and polymers (polyols, novolacs, resoles, phenalkamines, non-ionic surfactants, etc.), this affects UV stability, further highlighting cardanol's applicability in 1K polyurethanes, coatings and thermoplastics.

Accordingly, there is still a need to provide increased functionality to cardanol derivatives. The present disclosure provides novel monomers, oligomers, polymers and methods of making and using the same. The present application satisfies these needs as well as others that are readily apparent to one skilled in the art.

### BRIEF SUMMARY OF THE INVENTION

Cashew Nut Shell Liquid derivatives and methods for making and using the same are provided.

In one embodiment, a compound of formula I is provided:

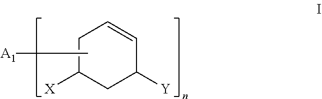

wherein:
A₁ is H; methylene; linear, branched and/or substituted alkyl; linear, branched and/or substituted aryl; linear, branched and/or substituted phenyl; linear, branched and/or substituted alkenyl; halogen
X and Y are each independently H and R
R is:

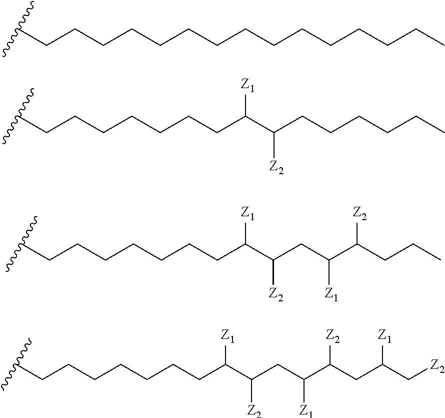

n is independently 0-50
Z₁ and Z₂ are each independently H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.

In other embodiment, a compound of formula II is provided:

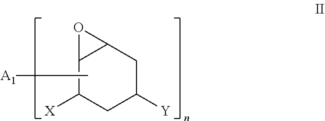

wherein:
A₁ is H; methylene; linear, branched and/or substituted alkyl; linear, branched and/or substituted aryl; linear, branched and/or substituted phenyl; linear, branched and/or substituted alkenyl; halogen
X and Y are each independently H and R
R is:

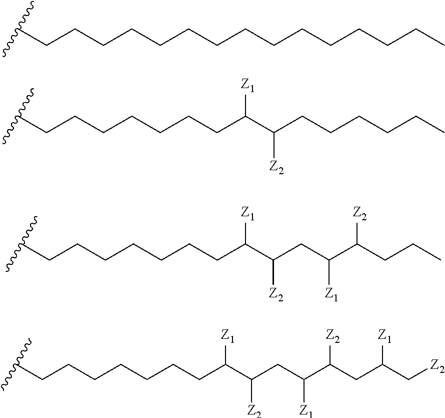

n is independently 0-50
Z₁ and Z₂ are each independently H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.

In another embodiment of the present invention, a compound of formula III is provided:

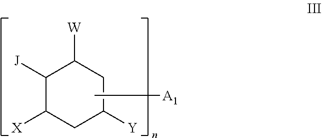

wherein:
A₁ is H; methylene; linear, branched and/or substituted alkyl; linear, branched and/or substituted aryl; linear, branched and/or substituted phenyl; linear, branched and/or substituted alkenyl; halogen
X and Y are each independently H and R
R is:

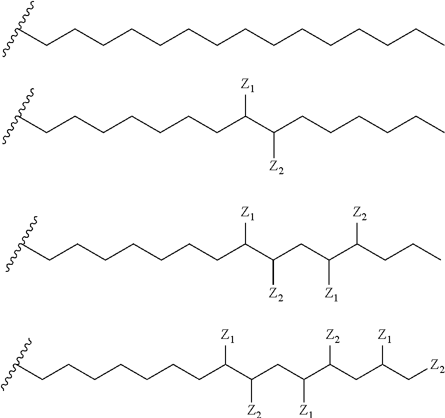

n is independently 0-50
Z₁ and Z₂ are each independently H; halogen; linear, branched and/or substituted alkyl; linear, branched and/or substituted aryl; linear, branched and/or substituted phenyl; linear, branched and/or substituted alkenyl; triazole; substituted triazole; linear alkoxy; branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.
J and W can be —H, —OH, —OCH₂(CHOCH₂), halogen, —NR₄R₅, —O—R₆, —O—CO—R₇, —O—CO—OR₈;
wherein R₄, R₅, R₆, R₇, R₈ are independently H, —CH₃, alkyl, vinyl, alkenyl, alkoxy, cycloalkyl, aryl, heteroaryl, and —O(CH₂CHR₉O)_{q}CH₂CHR₉OH; and
R₉ is H or CH₃
q is 1-100.

In some embodiments, methods for using compounds of formula I, II and III in antimicrobials, antioxidants, adhesives, coatings, corrosion retardants, composites, cosmetics, detergents, soaps, de-icing products, elastomers, food, flavors, inks, lubricants, oil field chemicals, tackifiers, prepolymer chain-extenders, rheology modifiers, electrical and electronic components (potting, castings, encapsulants), personal care products, polymers, structural polymers, engineered plastics, 3D printable polymers, UV/E-beam/cationic curable polymers, techno-polymers, rubbers, sealants, solvents, diluents, toughening agents, plasticizers, surfactants, varnishes, etc. are provided.

### BRIEF DESCRIPTION OF THE DRAWINGS

FIG. 1 illustrates an exemplary ¹H-NMIR of fully hydrogenated cardanol (3-pentadecyl-cyclohexanol).

FIG. 2 illustrates an exemplary GC chromatogram of a mixture of isomers, 3-pentadecyl-cyclohexene and 4-pentadecyl-cyclohexene.

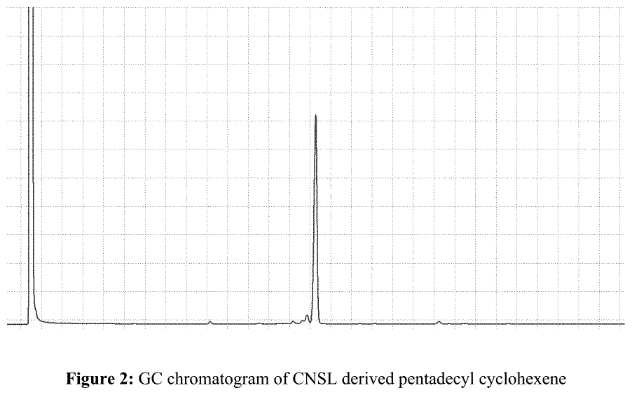

FIG. 3 illustrates an exemplary IR of a mixture of isomers, 3-pentadecyl-cyclohexene and 4-pentadecyl-cyclohexene.

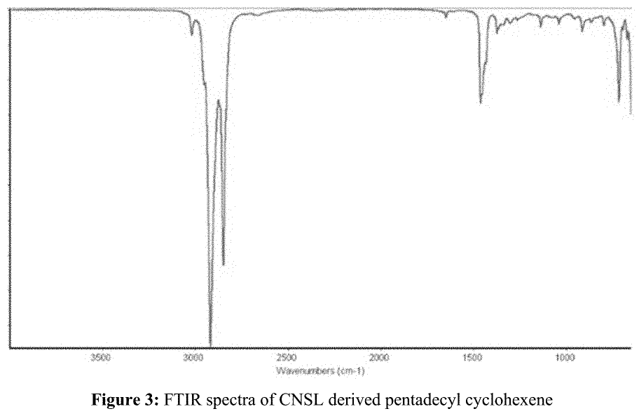

### DETAILED DESCRIPTION OF THE INVENTION

Embodiments described herein generally relate to Cashew Nut Shell Liquid derivatives and methods for making and using the same. More particularly, such embodiments relate to Cashew Nut Shell Liquid based compounds and derived polymers for making various products. Embodiments disclosed herein relate, in part, to the synthesis of cardanol-based derivatives through the full hydrogenation of any unsaturation present in their backbone, thus including all the double bonds present on cardanol's alkenyl C₁₅ side chain as well as of any aromatic ring belonging to cardanol on any other aromatic group.

In some embodiments, fully hydrogenated cardanol derivatives can be further transformed to pentadecyl-cyclohexene containing structures as mixture of isomers, namely substituted 3-pentadecyl-cyclohexene like derivatives and substituted 4-pentadecyl-cyclohexene like derivatives. These compounds represent useful substrates for further chemical transformations, which include but are not limited to, for example, etherification, alkylation, silylation, hydrogenation, ozonization.

In some embodiments, CNSL-derivatives that can be used as the starting materials in the presently described methods are cardanol, phenolated cardanol (as for instance NX-5266 available from Cardolite Corporation, USA, as a non limiting example), C₁₅-alkylated cardanol derivatives, C₁₅-maleated cardanol derivatives, C₁₅-aminated cardanol derivatives, cardanol-based hydrocarbon resins (like LITE 2100, LITE 2100R, both available from Cardolite Corporation, USA, as non-limiting examples), CNSL-distillation residues, oligomerized cardanol, cardanol-based resoles, cardanol based novolacs (as for instance NX-4005, NX-4004, NX-4001 available from Cardolite Corporation, USA, as non-limiting examples), silylated cardanol derivatives (as for instance NX-7303 available from Cardolite Corporation, USA, as a non-limiting example). Other CNSL-derivatives can also be used.

In other embodiments, these compounds can be used as versatile polymer building blocks, which include but are not limited to, for example, for the synthesis of alkyds, hydrolysable silanes, polyester diols and polyols, polyethers, amino-alcohols, epi sulfides, aziridines, carbonates, polyamides, halohydrines, epoxies, acrylates, that can be used in 1K and 2K adhesives, elastomers, coatings, epoxy formulations, polyurethanes, tackifiers, rheology modifiers, thermoplastics, reactive and non-reactive diluents, transformer oils, plasticizers, toughening agents, and the like.

As used herein, the term “alkyl” whether used alone or as part of another group, refers to a substituted or unsubstituted aliphatic hydrocarbon chain and includes, but is not limited to, straight and branched chains containing from 1 to 20 carbon atoms, preferably from 2 to 20, from 1 to 10, from 2 to 10, from 1 to 8, from 2 to 8, from 1 to 6, from 2 to 6, from 1 to 4, from 2 to 4, from 1 to 3 carbon atoms, unless explicitly specified otherwise. Illustrative alkyl groups can include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl- 1-butyl, 3-methyl-1-butyl, 2-methyl-3-butyl, 2-methyl- 1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkenyl” whether used alone or as part of another group, refers to a substituted or unsubstituted aliphatic hydrocarbon chain and includes, but is not limited to, straight and branched chains having 2 to 8 carbon atoms and containing at least one carbon-carbon double bond.

As used herein, the term “alkynyl” whether used alone or as part of another group, refers to a substituted or unsubstituted aliphatic hydrocarbon chain and includes, but is not limited to, straight and branched chains having 1 to 6 carbon atoms and containing at least one carbon-carbon triple bond.

As used herein, the term “alkoxy” whether used alone or as part of another group, refers to alkyl-O— wherein alkyl is hereinbefore defined.

As used herein, the term “cycloalkyl” whether used alone or as part of another group, refers to a monocyclic, bicyclic, tricyclic, fused, bridged or spiro monovalent saturated hydrocarbon moiety, wherein the carbon atoms are located inside or outside of the ring system. Any suitable ring position of the cycloalkyl moiety may be covalently linked to the defined chemical structures. Illustrative cycloalkyl groups can include, but are not limited to, cyclopropyl, cyclopropylmethyl, cyclobutyl, cyclopentyl, cyclohexyl, cyclohexylmethyl, cyclohexylethyl, cycloheptyl, norbornyl, adamantly, spiro[4,5]decanyl, and homologs, isomers and the alike.

As used herein, the term “aryl” whether used alone or as part of another group, refers to an aromatic carbocyclic ring system having 6 to 14 carbon atoms, preferably 5 to 10 carbon atoms, optionally substituted with 1 to 3 substituents independently selected from halogen, nitro cyano, hydroxy, alkyl, alkenyl, alkoxy, cycloalkyl, amino, alkylamino, dialkylamino, carboxy, alkoxycarbonyl, haloalkyl, and phenyl.

As used herein, the term “phenyl” whether used alone or as part of another group, refers to a substituted or unsubstituted phenyl group.

As used herein, the term “heteroaryl” whether used alone or as part of another group, refers to a 5 to 10 membered aryl heterocyclic ring, which contains from 1 to 4 heteroatoms selected from the group consisting of O, N and S atoms in the ring and may be fused with a carbocyclic or heterocyclic ring at any possible position.

As used herein, the term “heterocycloalkyl” whether used alone or as part of another group, refers to a 5 to 7 membered saturated ring containing carbon atoms and from 1 to 2 heteroatoms selected from the group consisting of O, N and S atoms.

As used herein, the term “halogen or halo” refers to fluoro, chloro, bromo or iodo.

As used herein, the term “haloalkyl” whether used alone or as part of another group, refers to an alkyl as hereinbefore defined, independently substituted with 1 to 3, F, Cl, Br or I.

As used herein, the term “about” refers that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, in phrase “about X to Y,” is the same as “about X to about Y,” that is the term “about” modifies both “X” and “Y.”

As used herein, the term “compound” refers to salts, solvates, complexes, isomers, stereoisomers, diastereoisomers, tautomers, and isotopes of the compound or any combination thereof.

As used herein, the term “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are used in their inclusive, open-ended, and non-limiting sense.

Cashew Nut Shell Liquid (CNSL) is a well-known non-edible natural oil obtained as a by-product of the Anacardium occidentale nut. CNSL is a non-food chain industrial oil found in the honeycomb structure of the cashew (Anacardium occidentale) nutshell, typically considered a by-product of the cashew nut industry. CNSL consists of a mixture of different chemical moieties (anacardic acid, cardanol, 2-methyl-cardol, cardol), all of them characterized by the presence of a C₁₅ side chain in the meta-position of the aromatic ring. This side chain contains a number of unsaturation from 0 to 3, with an average number of 2 double bonds. The main product isolated by vacuum distillation of CNSL under proper conditions is cardanol, which is an important chemical derived by decarboxylation of anacardic acid, as the primary component of CNSL. Cardanol is essentially, a meta-substituted phenol ring with mono-, di-, tri- unsaturated and saturated long 15-carbon chain, as shown below:

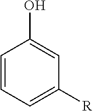

where R can be, for example:

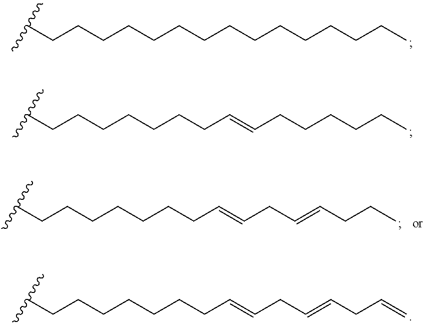 [SMILES: *CCCCCCCCCCCCCCC ; *CCCCCCC/C=C/CCCCCC ; *CCCCCCC/C=C/C/C=C/CCC ; *CCCCCCC/C=C/C/C=C/CC=C]

Cardanol is characterized by a peculiar chemical structure: the aromatic provides excellent rigidity and thermal stability, while the C₁₅ unsaturated aliphatic side chain at the meta position imparts outstanding hydrophobicity and flexibility. The unsaturations on the C₁₅ side chain can be further derivatized to useful chemicals, which can be used in coatings, adhesives, antioxidants, elastomers, food, flavors, lubricants, polymers, rubbers, sealants etc. applications.

In some embodiments, cardanol has a purity from about 80% to about 99.9%, and preferably from about 95% to about 99.5%. Cardanol was treated under reductive conditions in order to hydrogenate all the double bonds on the C₁₅ side chain as well as on the aromatic ring, yielding 3-pentadecyl-cyclohexanol in high purity and yield. Hydrogenation was carried on at a pressure from about 5 Bar to about 50 Bar and preferably from about 15 Bar to about 25 Bar and a temperature at about 30-200° C. and preferably at about 100-180° C. in presence of a catalyst. Illustrative catalysts can include, but are not limited to, Pd/C, Pd(OH)₂, Pd/Al₂O₃, Pd/NaY, Ru-PVP NPs, Ru/C, RuC;₃, Ni and Ni Raney or any combination thereof. The dosage level of the catalyst is about 1-10 w/w and preferably about 3-5% w/w, with or without the presence of a Lewis acid at about 5-20 mol % and preferably 8-12 mol %, without any limitations thereof. Illustrative solvents can include, but are not limited to, methanol, ethanol, isopropanol, n-butanol, hexane, cyclohexane, cyclopentane, N-methylpyrrolidone, N,N-dimethylformamide, dimethylsulfoxide, methylene chloride, chloroform, carbon tetrachloride, tetrahydrofuran, water or any combination thereof. The chemical structure of fully hydrogenated cardanol is shown below:

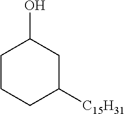

Cyclohexene is a well-known molecule, typically used as building block in the pharma industry or as a precursor of adipic acid, malic acid, epoxide, diol and other useful downstream products, like elastomers, adhesives, silanes, waterproof coatings (Vafaeezadeh, M.; Mahmoodi Hashemi, M. Chemical Engineering Journal 2013, 221, 254-257; Yoshikazu, T.; Tokitaka, K. US0018223; Reddy, A. S.; Chen, C. Y.; Chen, C. C.; Chien, S. H.; Lin, C. J.; Lin, K. H.; Chen, C. L.; Chang, S. C. J. Molecular Catalysis A: Chemical, 2010, 318, 60-67; Noritaka, M.; Lyon, D. K.; Finke, R. G. US5250739; Luque, R.; Badamali, S. K.; Clark, J. H.; Fleming, M.; Macquerrie, D. J. Applied Catalysis A. General, 2008, 341, 154-159; Vafaeezadeh, M.; Hashemi, M. M. Catalysis Communications 2014, 43, 169-172).

The conversion of cyclohexanol into cyclohexene is a well-known process (Sandler, S. R.; Karo, W. OLEFINS. Sourcebook of Advanced Organic Laboratory Preparations, 1992, 16-23; Aizawa, H.; Kuroda, A.; Minaga, M.; Ohnishi, K.; Matsuhisa, S.; US3974232; Had, N.; Halimaton, H. Reaction Kinetics and Catalysis Letters, 1999, 66, 33-38; Athappan, R., Srivastava, R. D. AIChE Journal, 1980, 26(3), 517-521; Akiya N.; Savage, P. E. Ind. Eng. Chem. Res. 2001, 40, 1822-1831; Sandler, S. R.; Karo, W. Sourcebook of Advanced Organic Laboratory Preparations 1992, Chapter 2, 16-23). However, cyclohexene is usually recovered from petro-based raw materials and is also characterized by health and safety limitations.

On this basis, 3-pentadecyl-cyclohexanol represents a suitable substrate for the recovery of a CNSL-based, fully bio-derived cyclohexene.

In some other embodiments, following the same conditions as for cyclohexene, fully hydrogenated cardanol 3-pentadecyl-cyclohexanol was dehydrated to a substituted alkyl cyclohexene as a mixture of isomers, namely 3-pentadecyl-cyclohexene and 4-pentadecyl-cyclohexene, as shown below:

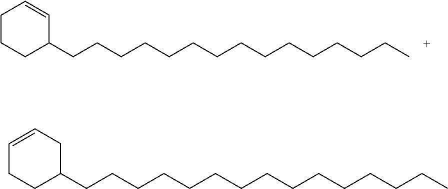

As a non-limiting example, 3-Pentadecyl-cyclohexanol was treated with phosphoric acid (with a concentration between 10% and 99%, and preferably 49.5-99%, and more preferably 85%). The number of phosphoric acid moles per each mole of 3-pentadecyl-cyclohexanol was between 0.1 and 20 and more preferable between 0.3 and 10 and even more preferably between 0.4 and 1. Reaction was carried on at about 100° C. and 350° C., preferably at about 150° C. and 200° C., under vacuum set between 0.5 mmHg and 100 mmHg and more preferably at 30 mmHg. The resulting alkyl-substituted cyclohexene derivatives, 3-pentadecyl-cyclohexene and 4-pentadecyl-cyclohexene can be eventually further purified by distillation, if needed, or used as such.

Other inorganic compounds suitable for the conversion of 3-pentadecyl-cyclohexanol to pentadecyl-cyclohexenes as a mixture of isomers 3-pentadecyl-cyclohexene and 4-pentadecyl-cyclohexene) are sulfuric acid, Al₂O₃, Al₂O₃/P₂O₅/pyridine, POCl₃/pyridine or any combination thereof.

Among the others, one major advantage of the present invention includes using 3-pentadecyl-cyclohexanol as the starting substrate to recover cardanol-derived cyclohexene like structures (3-pentadecyl-cyclohexene and 4-pentadecyl-cyclohexene) in high purity and yields.

A further advantage of the present invention is its applicability to other cardanol derivatives, including cardanol monomers with substituents on the C₁₅ side chain, oligomerized cardanol, polymerized cardanol, CNSL-distillation residues, cardanol-containing structures bearing unmodified phenoxy groups, their combinations and without any limitation thereof.

In some other embodiments, following the same conditions as for pentadecyl-cyclohexene, fully hydrogenated cardanol-derivatives are dehydrated to substituted alkyl cyclohexene containing structures as a mixture of isomers, as shown below:

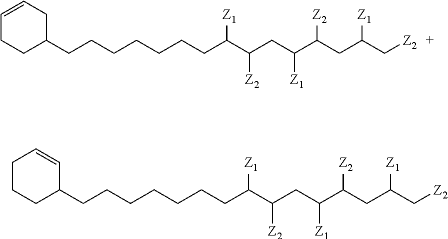

wherein
Z₁ and Z₂ are each independently H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.

Without being bound to any particular theory, one advantage of the methods and compounds described herein is the possibility to generate novel monomers with different types of functionalities that can be further reacted with other raw materials. For example, 3-pentadecyl-cyclohexene and 4-pentadecyl-cyclohexene can be used as such (e.g. diluents, plasticizers) and as starting materials to synthesize novel monomers, which include, but not limited to, epoxides, episulfides, aziridines, diols, amino-alcohols, silanes, polyesters, polyethers, polycarbonates, acrylates, methacrylates, hydrocarbon resins.

One further advantage of the present invention is the applicability of the methods herein described to different types of cardanol derivatives, allowing the recovery of new monomers and polymer building blocks characterized by a cycloaliphatic backbone and a wide range of functionalities and physico-chemical properties.

Accordingly, in some embodiments, a compound of formula I is provided:

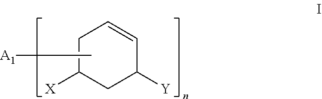

wherein
A₁ is H; methylene; linear, branched and/or substituted alkyl; linear, branched and/or substituted aryl; linear, branched and/or substituted phenyl; linear, branched and/or substituted alkenyl; halogen
X and Y are each independently H and R
R is:

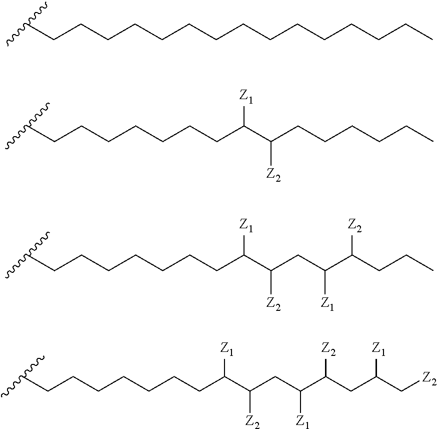

n is independently 0-50
Z₁ and Z₂ are each independently H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.

In some embodiments, methods for preparing the compound of formula I are provided. In some embodiments, the methods comprise hydrogenating cardanol or a cardanol derivative with hydrogen gas in the presence of at least one catalyst and an optional solvent; maintaining the reaction temperature from about 160° C. to about 180° C. for a period of about 5 hours to about 15 hours and the pressure from about 10 Bar to about 25 Bar; removing the catalyst via filtration to produce a fully hydrogenated product; adding the fully hydrogenated product to a mixture of at least one oxidant and at least one catalyst for a period of about 1 hour to about 3 hours at a temperature below 55° C.; stirring the reaction mixture at temperature from about 60° C. to about 80° C. for a period of about 4 hours to about 20 hours; separating the reaction mixture into an aqueous phase and an organic phase; removing the aqueous phase; adding an air to the organic phase at a temperature from about 80° C. to about 90° C.; washing the organic phase with water; and applying vacuum to produce the compound.

The hydrogenation of cardanol or a cardanol derivative can be carried out at a temperature from a low of about 30° C. to a high of about 200° C. For example, the temperature can be from about 50° C. to about 190° C., from about 100° C. to about 185° C., or from about 160° C. to about 180° C. In some embodiments, the temperature is from about 160° C. to about 180° C. The hydrogenation of cardanol or a cardanol derivative can be carried out at a pressure from a low of about 5 Bar to a high of about 50 Bar. For example, the pressure can be from about 7 Bar to about 40 Bar, from about 9 Bar to about 30 Bar, or from about 10 Bar to about 25 Bar. In some embodiments, the pressure is from about 10 Bar to about 25 Bar. The length of time for the hydrogenation of cardanol or a cardanol derivative can be from a low of about 2 hours to a high of about 15 hours. For example, the time can be from about 7 hours to about 13 hours, from about 9 hours to about 11 hours, from about 9.5 hours to about 10 hours. In some embodiments, the time is about 10 hours.

In some embodiments, the dehydration of cardanol derived pentadecyl-cyclohexanol containing structures can be carried out at a temperature from a low of about 100° C. to a high of about 350° C. For example, the temperature can be from about 100° C. to about 160° C., from about 110° C. to about 180° C., or from about 160° C. to about 350° C. The number of dehydrating agent moles per each mole of substrate is from about 0.1 to about 20 and more preferable between 0.3 and 10 and even more preferably between 0.4 and 1. For example, the moles can be from about 0.3 to about 5, from about 2.5 to about 4 or from about 3 to about 6. In some embodiments, the moles are from about 0.3 to about 0.5.

Illustrative hydrogenation catalysts can include, but are not limited to, Pd/C, Pd(OH)₂, Pd/Al₂O₃, Pd/NaY, Ru-PVP NPs, Ru/C, RuCl₃, Ni and Ni Raney or any combination thereof.

Illustrative dehydrating agents can include, but are not limited to, phosphoric acid, sulfuric acid, Al₂O₃, Al₂O₃/P₂O₅/pyridine, POCl₃/pyridine or any combination thereof.

In some embodiments, cardanol has a purity from about 80% to about 99.5% and preferably from about 95% to about 99.5%.

In some embodiments, cardanol-derivatives as suitable starting materials in the presently described methods can be selected among C₁₅-phenolated cardanol, C₁₅-alkylated cardanol derivatives, C₁₅-maleated cardanol derivatives, C₁₅-aminated cardanol derivatives, cardanol-based hydrocarbon resins, CNSL-distillation residues, oligomerized cardanol, cardanol-based resoles, cardanol based novolacs, silylated cardanol derivatives, their combinations and without any limitation thereof.

Another advantage of the methods and compounds described herein is the validation of cardanol-derived pentadecyl-cyclohexene like structures as diluents, plasticizers and toughening agents for both thermosetting and thermoplastic materials. In some embodiments, cardanol-derived pentadecyl-cyclohexene like structures are used as polymer building blocks, for the synthesis of a variety of functional monomers and polymers. Cardanol-derived pentadecyl-cyclohexene like structures can be used as single source of reactive functional groups, namely their double bond. In some embodiments, these cardanol-derivatives can be used from 1% to 100% weight percent of the formulation or in combination with other petro-derivatives or bio-derived unsaturated raw materials. However, all these raw materials amounts are only exemplary figures and are not intended to be limiting. Other combinations can be used and can be adjusted depending on the specific reagents used.

In some embodiments, a compound of formula II is provided as:

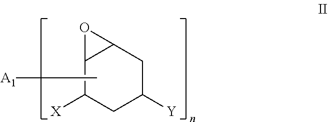

wherein:
A₁ is H; methylene; linear, branched and/or substituted alkyl; linear, branched and/or substituted aryl; linear, branched and/or substituted phenyl; linear, branched and/or substituted alkenyl; halogen
X and Y are each independently H and R
R is:

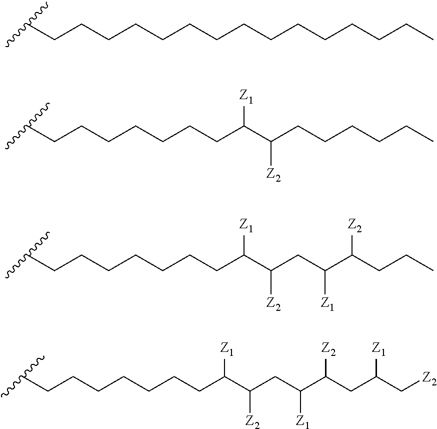

n is independently 0-50
Z₁ and Z₂ are each independently H; halogen; linear, branched and/or substituted alkyl; linear, branched and/or substituted aryl; linear, branched and/or substituted phenyl; linear, branched and/or substituted alkenyl; triazole; substituted triazole; linear alkoxy; branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;

R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.

In some embodiments, the compound of formula II is provided as a mixture of isomers, as shown below:

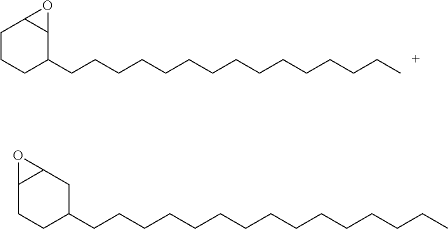

In some embodiments, the compound of formula II is provided as a mixture of isomers, as shown below:

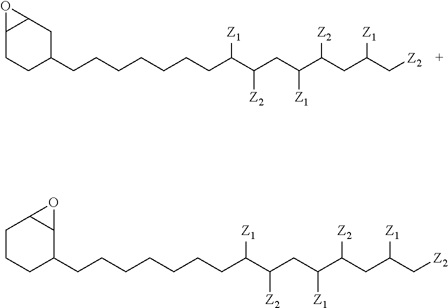

In some embodiments, methods for preparing the compound of formula II are provided. In some embodiments, the methods comprise reacting a mixture of cardanol-derived pentadecyl-cyclohexene like structures with an oxidizing agent; stirring the reaction mixture at a temperature from 50° C. to 70° C. for a period of about 9 to about 11 hours; separating the aqueous phase from the organic phase; neutralizing the organic phase and isolating the target epoxy derivatives as a mixture of isomers.

In certain embodiments, the temperature of the reaction between a mixture of cardanol-derived pentadecyl-cyclohexene like structures and an oxidizing agent is from about −15° C., 0° C., 5° C., 10° C., 15° C., 25° C., 35° C., 45° C., 60° C. or 75° C. Any of these values may be used to define a range for the temperature for the reaction between a mixture of diacids and a diol compound. For example, the temperature may range from about −25° C. to about 85° C., from about −10° C. to about 45° C., or from about 35° C. to about 75° C. In some embodiments, the temperature is from about 55° C. to about 65° C. In certain embodiments, the reaction time between a mixture of cardanol-derived pentadecyl-cyclohexene like structures and an oxidizing agent is about 2 hour, 3 hours, 5 hours, 8 hours, 10 hours, 13 hours, 15 hours, 18 hours or 20 hours. Any of these values may be used to define a range for the length of time for the reaction between a mixture of cardanol-derived pentadecyl-cyclohexene like structures and an oxidizing agent. For example, the length of time may range from a low of about 2 hours to a high of about 20 hours, from about 3 hours to about 18 hours, from about 5 hours to about 15 hours. In some embodiments, the time is from about 10 hours to about 14 hours.

Illustrative oxidants can include, but are not limited to, hydrogen peroxide/formic acid mixture, hydrogen peroxide/acetic acid mixture, tri-chloro-acetonitrile/hydrogen peroxide, meta-chloro per-benzoic acid, Oxone® or any combination thereof.

Illustrative solvents can include, but are not limited to, toluene, xylene, methylene chloride, acetone, chloroform, diethyl ether, buffered tert-butanol, acetonitrile, dioxane, dimethyl-ether, or any combination thereof.

Another advantage of the methods and compounds described herein is the validation epoxy compounds prepared from cardanol-derived pentadecyl-cyclohexene like structures, as versatile substrates for the synthesis of novel monomers and as polymer building blocks, for the synthesis of a variety of functional monomers and polymers. These isomers can be used as single source of epoxy groups. In some other embodiments, these cardanol-derived epoxy-functional derivatives can be used from 1% to 100% weight percent of the formulation or in combination with other petro-derivatives or bio-derived unsaturated raw materials. However, all these raw materials amounts are only exemplary figures and are not intended to be limiting. Other combinations can be used and can be adjusted depending on the specific reagents used.

In some embodiments, a compound of formula III is provided as:

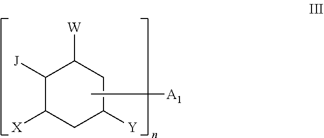

wherein:
A₁ is H; methylene; linear, branched and/or substituted alkyl; linear, branched and/or substituted aryl; linear, branched and/or substituted phenyl; linear, branched and/or substituted alkenyl; halogen
X and Y are each independently H and R
R is:

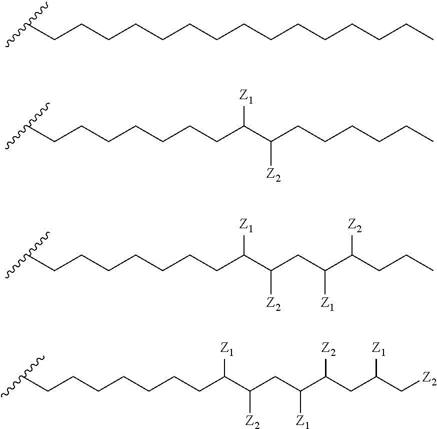

n is independently 0-50
Z₁ and Z₂ are each independently H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.
J and W can be —H, —OH, —OCH₂(CHOCH₂), halogen, —NR₄R₅, —O—R₆, —O—CO—R₇, —O—CO—OR₈;
wherein R₄, R₅, R₆, R₇, R₈ are independently H, —CH₃, alkyl, vinyl, alkenyl, alkoxy, cycloalkyl, aryl, heteroaryl, and —O(CH₂CHR₉O)ₙCH₂CHR₉OH; and
R₉ is H or CH₃
n is 1-100.

In some embodiments, the compound of formula III is provided as:

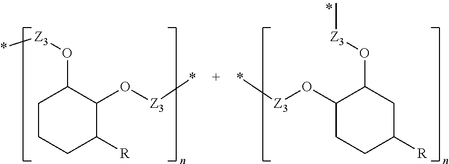

wherein
R is:

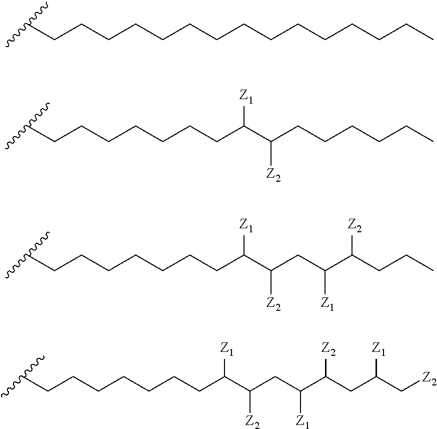

Z₁ and Z₂ are each independently H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ and R₃ are each independently a saturated or unsaturated hydrocarbon chain of 1-20 carbon atoms, or is a hydrolysable group selected from chloro, methoxy, propoxy, methylalkoxy, acetoxy, silazanes, oximes, and the like;
J and W can be —H, —OH, —OCH₂(CHOCH₂), halogen, —NR₄R₅, —O—R₆, —O—CO—R₇, —O—CO—OR₈;
wherein R₄, R₅, R₆, R₇, R₈ are independently H, —CH₃, alkyl, vinyl, alkenyl, alkoxy, cycloalkyl, aryl, heteroaryl, and —O(CH₂CHR₉O)ₚCH₂CHR₉OH; and
R₉ is H or CH₃
Z₃ is H, —CH₃, alkyl, vinyl, alkenyl, alkoxy, cycloalkyl, aryl, phenyl, heteroaryl, —CH₂(CHOCH₂) and —O(CH₂CHR₉O)ₚCH₂CHR₉OH and
n is 1-50
p is 0-100

In some embodiments, the compound of formula III is provided as:

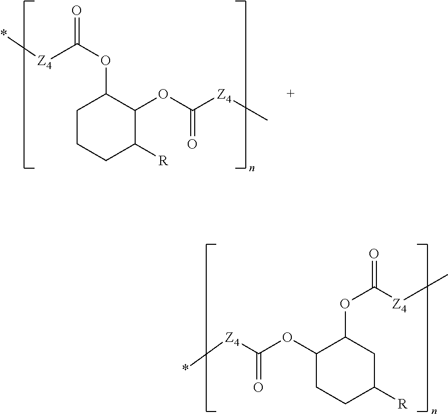

wherein
R is:

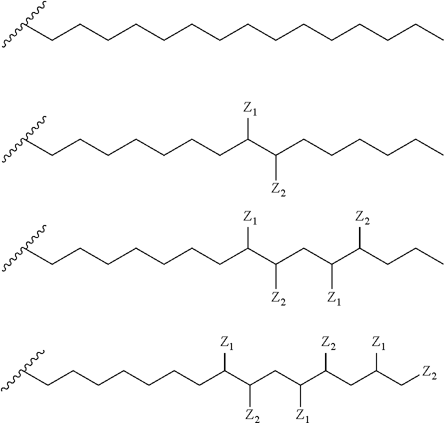

Z₁ and Z₂ are each independently H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
R₁ is independently a hydrolysable group selected from chloro, methoxy, propoxy, methylalkoxy, acetoxy, silazanes, oximes, and the like;
R₂ and R₃ are each independently a saturated or unsaturated hydrocarbon chain of 1-20 carbon atoms, or is hydrolysable group selected from chloro, methoxy, propoxy, methylalkoxy, acetoxy, silazanes, oximes, and the like;
J and W can be —H, —OH, —OCH₂(CHOCH₂), halogen, —NR₄R₅, —O—R₆, —O—CO—R₇, —O—CO—OR₈;
wherein R₄, R₅, R₆, R₇, R₈ are independently H, —CH₃, alkyl, vinyl, alkenyl, alkoxy, cycloalkyl, aryl, heteroaryl, and —O(CH₂CHR₉O)ₚCH₂CHR₉OH; and
R₉ is H or CH₃
Z₄ is —OCH₃, alkyl, vinyl, alkenyl, alkoxy, cycloalkyl, aryl, phenyl, heteroaryl, —CH₂(CHOCH₂) and —O(CH₂CHR₉O)ₚCH₂CHR₉OH, —CH═CH₂, —CH(CH₃)═CH₂
n is 1-50
p is 0-100

In some embodiments, the compound of formula III is provided as:

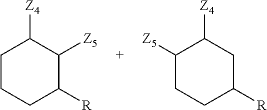

wherein
R is:

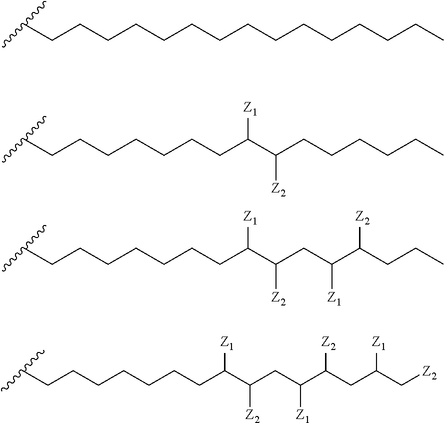

Z₁ and Z₂ are each independently H; halogen; linear, branched and/or substituted alkyl; linear, branched and/or substituted aryl; linear, branched and/or substituted phenyl; linear, branched and/or substituted alkenyl; triazole; substituted triazole; linear alkoxy; branched alkoxy; —CHCH₃(CH₂)ₘSiR₁R₂R₃
R₁ is independently a hydrolysable group selected from chloro, methoxy, propoxy, methylalkoxy, acetoxy, silazanes, oximes, and the like;
R₂ and R₃ are each independently a saturated or unsaturated hydrocarbon chain of 1-20 carbon atoms, or is hydrolysable group selected from chloro, methoxy, propoxy, methylalkoxy, acetoxy, silazanes, oximes, and the like;
J and W can be —H, —OH, —OCH₂(CHOCH₂), halogen, —NR₄R₅, —O—R₆, —O—CO—R₇, —O—CO—OR₈;
wherein R₄, R₅, R₆, R₇, R₈ are independently H, —CH₃, alkyl, vinyl, alkenyl, alkoxy, cycloalkyl, aryl, heteroaryl, and —O(CH₂CHR₉O)ₘCH₂CHR₉OH; and
R₉ is H or CH₃
Z₄ and Z₅ are H, OH, alkyl, vinyl, alkenyl, alkoxy, cycloalkyl, aryl, phenyl, heteroaryl, halogen, —NR₁₀R₁₁, —O—CO—OR₉
wherein R₁₀ and R₁₁, are independently H, —CH₃, alkyl, vinyl, alkenyl, alkoxy, cycloalkyl, aryl, heteroaryl, and —O(CH₂CHR₉O)ₙCH₂CHR₉OH and
R₉ is H or CH₃
n is 1-100.

In some embodiments, methods for preparing a compound of formula III are provided. In some embodiments, the methods comprise reacting a mixture of epoxy compounds prepared from cardanol-derived pentadecyl-cyclohexene like structures with a nucleophile compound or nucleophile compounds; stirring the reaction mixture at a temperature from about 10° C. to about 250° C. for a period of about 0.5 hours to about 4 hours; adding at least one optional catalyst to the reaction mixture; maintaining the reaction mixture at a temperature from about 15° C. to about 250° C. for a period of about 10 hours to about 20 hours; and cooling the reaction mixture at a room temperature to produce the derivative. In certain embodiments, the temperature of the reaction between a mixture of pentadecyl-cyclohexenes derived epoxy isomers and nucleophile is about 10° C., 25° C., 60° C., 90° C., 120° C., 180° C., 200° C., 220° C., or 250° C. In certain embodiments, the reaction is carried on at atmospheric pressure or under pressure, as for example, from about 1.5 to about 10 bars. Any of these values may be used to define a range for the temperature for the reaction between a mixture of epoxies and a nucleophile compound. For example, the temperature may range from about 25° C. to about 100° C., from about 120° C. to about 200° C., or from about 150° C. to about 180° C. In certain embodiments, the reaction time between epoxy compounds prepared from cardanol-derived pentadecyl-cyclohexene like structures and a nucleophile is about 1 hour, 3 hours, 5 hours, 8 hours, 10 hours, 13 hours, 15 hours, 18 hours or 20 hours. Any of these values may be used to define a range for the length of time for the reaction between epoxy compounds prepared from cardanol-derived pentadecyl-cyclohexene like structures and a nucleophile. For example, the length of time may range from a low of about 2 hours to a high of about 20 hours, from about 3 hours to about 18 hours, from about 5 hours to about 15 hours. In some embodiments, the time is from about 10 hours to about 14 hours.

Illustrative nucleophiles can include, but are not limited to, diols, polyols, amines, mono-carboxylic acid, dicarboxylic acids, hydrogen, ammonia, primary amines, secondary amines, hydrogen disulfide, sodium hydrogen sulfide, inorganic acids, or any combination thereof.

In some embodiments, illustrative diols and polyols can include, but are not limited to, neopentyl glycol; 2-methyl-1,3 -propane-diol; 2-methy-2,4-pentane-diol; 2-butyl-2-ethyl-1,3-propanediol; 2-ethyl-1,3-hexane diol; 2,4-diethyl-1,5-pentane diol; 1,2-propylene glycol; di-propylene glycol; ethylene glycol; diethylene glycol; triethylene glycol; 1,3-propane glycol; butylene glycols; 1,2-cyclohexanediol; polyoxyalkylene polyols; glycerol; 1,1,1-trimethylolpropane; 1,1,1-trimethylolethane; pentaerythritol or any combination thereof.

In some embodiments, illustrative organic acid compounds can be, for example, aromatic di-acids like phthalic acid; terephthalic acid; isophthalic acid; phthalic anhydride; dimethyl terephthalate; dimethyl phthalate; dimethyl isophthalate; polyethylene terephathalate; L-Lactide; 2,5-furandicarboxylic acid; trimellitic anhydride; derivatives thereof, and combinations thereof. Di-acids can be selected among aliphatic ones, too, as for example, but not-limited to, fatty acids; mono-carboxylic acids of 1-30 carbons; adipic acid; succinic acid; glutaric acid; azelaic acid; sebacic acid; citric acid; trimethylolpropionic acid; dimer acids; trimer acids of fatty acid origin; acrylic acid; methacrylic acid; acetic acid; formic acid, or mixtures thereof.

Illustrative amines can include, but are not limited to, hydroxylamine; hydroxylamine hydrochloride; diethylenetriamine; tetraethylenepentamine; 1-(1-phenylcyclopentyl)methylamine; 1-hexanamine; ethylenediamine; 2,4-dimethylpentan-3-amine; 2-isopropylaminoethylamine; 2-methylbutan-2-amine; 2N-(3 -aminopropyl)-4-aminobutanal; N-isopropyl-2-methylpropan- 1,2-diamine; isophoronediamine; sec-butylamine; tert-butylamine; amantadine; butan-1-amine; cyclohexane-1,2-diamine; cyclohexylamine; cyclopropylamine; dicyclohexylamine; ethylamine; isopentylamine; isopropylamine; octadecan-1-amine; octan-1-amine; pentan-1-amine; pentan-3 -amine; dimethylamine; diethylmethylamine; 2-aminoethanol; aniline; m-bromoaniline; 2-chloroaniline; 3,5-dichloroaniline; methylamine; 4-methoxyaniline; 3-Nitroaniline; 4-nitroaniline; 4-trifluoromethylaniline; 2,2′-dichloro-4,4′-methylenedianiline (MOCA); 2,4,5-trimethylaniline; 2-methoxyaniline,o-Anisidine; 2-naphthylamine; 3,3′-dichlorobenzidine 3,3′-dichlorobiphenyl-4,4′-ylenediamine; 3,3′-dimethoxybenzidine o-dianisidine; 3,3′-dimethylbenzidine 4,4′-bi-o-toluidine; 4,4′-methylenedi-o-toluidine; 4,4′-oxydianiline; 4,4′-thiodianiline; m-Xylylenediamine; 4,4′-diaminodiphenylmethane (MDA); 4-Aminoazobenzene; 4-chloro-o-toluidine; 4-chloroaniline; 4-methoxy-m-phenylenediamine; -methyl-m-phenylenediamine (toluene-2,4-diamine); 5-nitro-o-toluidine; 6-methoxy-m-toluidine (p-cresidine); benzidine; biphenyl-4-ylamine,4-aminobiphenyl xenylamine; o-aminoazotoluene,4-amino-2′,3-dimethylazobenzene,4-o-tolylazo-o-toluidine; o-toluidine,2-aminotoluene; tetramethylene diamine; pentamethylene diamine; hexamethylene diamine; decamethylene diamine or any combination thereof.

Illustrative examples of inorganic acids can be sulfuric acid; phosphoric acid; hydrochloric acid; perchloric acid; methane-sulfonic acid; hydrofluoric acid; tetrafluoroboric acid or any combination thereof.

Other aspects and advantages of these novel cardanol-derived products, as well as their combinations, will be apparent to those skilled in the art. Experimental details are provided in the following examples, which are provided by way of illustration only and should not be construed to limit the disclosure or the appended claims.

### EXAMPLES

In order to provide a better understanding of the foregoing discussion, the following non-limiting examples are offered. Although the examples may be directed to specific embodiments, they are not to be viewed as limiting the invention in any specific respect. All parts, proportions, and percentages are by weight unless otherwise indicated.

### Example 1

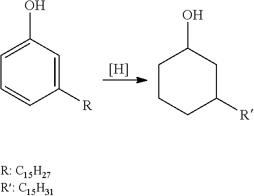

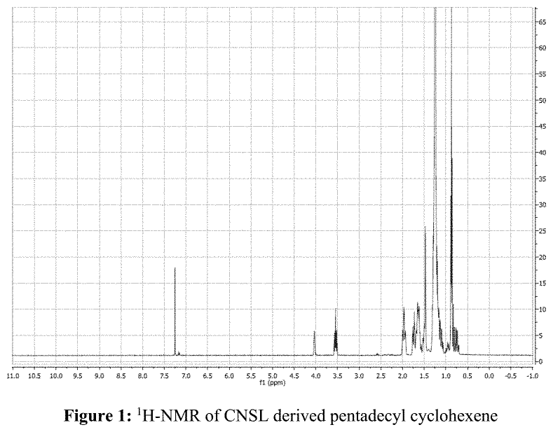

In a Parr reactor, cardanol (300 g; 1 mol) was mixed with Ni catalyst (3% w/w with respect to substrate). The temperature of the reaction mixture was then raised to 170° C. under hydrogen atmosphere (24 Bar) and maintained for 10 hours. The catalyst was removed via filtration over Celite° recovering 3-pentadecyl-cyclohexanol, as a white solid, 85% yield, m.p. 49-51° C. The structure of 3-pentadecyl-cyclohexanol was confirmed and characterized by ¹H-NMR spectrum (FIG. 1).

### Example 2

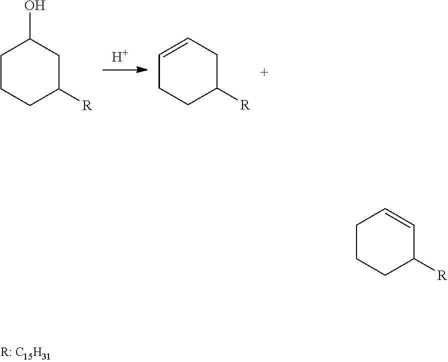

To a round bottom flask equipped with mechanical overhead mixer, thermocouple, and distillation apparatus was added 3-pentadecyl-cyclohexanol (308 g, 1 mol) and concentrated phosphoric acid as an 85% aqueous solution (57 g, 0.5 mol). This mixture is heated to around 90° C. and water is removed under vacuum. Then, while still under vacuum, the mixture is heated to 140-150° C. and the water of reaction is removed until the reaction is complete (confirmed by GC). Then, strong vacuum is applied and the product is further heated to initiate distillation of 3- and 4-pentadecyl cyclohexene as a clear liquid mixture of isomers in greater than 90% yield.

### Example 3

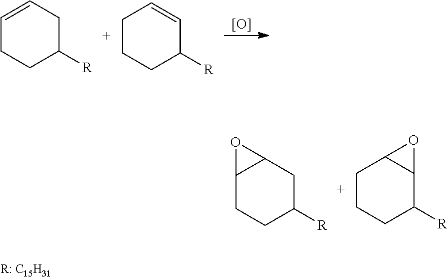

To a round bottom flask equipped with mechanical overhead mixer, thermocouple, and reflux condenser was added mixture of isomers of 3- and 4-pentadecyl cyclohexene (292 g, 1 mol), formic acid (27 g, 0.6 mol), deionized water (72 g), and a 50% aqueous solution of hydrogen peroxide (204 g, 3 mol). This mixture was heated to around 50° C. at which point a mild exotherm takes over and the batch is maintained with intermittent external cooling below 60° C. until the exotherm subsides. Then, the reactor is heated at 55° C. for 18 hours until complete conversion of substrate (confirmed by GC). Mixing is stopped and the contents of the reactor are allowed to separate completely before discarding the aqueous phase. Then the organic product is washed twice with water before vacuum dehydration to remove traces of water resulting in the final product 3- and 4-pentadecyl cyclohexene oxide as a pale yellow liquid mixture of isomers in 99% yield.

### Example 4

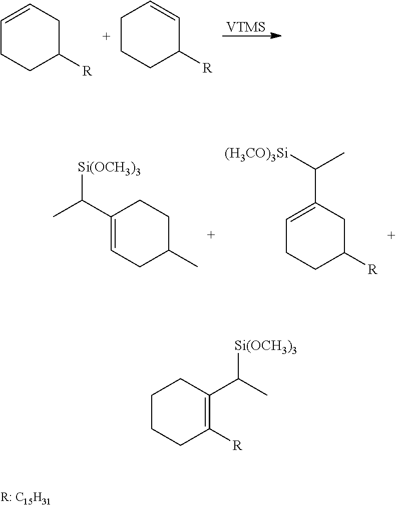

Pentadecyl-cyclohexene isomers 3-pentadecyl-cyclohexene and 4-pentadecyl-cyclohexene (292 g, 1 mol) was mixed with vinyltrimethoxysilane (207 g, 1.4 moles) in a high-pressure stainless-steel PARR reactor (PARR Instrument Company, IL, USA). Luperox-101 (6 g, 1% w/w) was then added and the reaction mixture then purged with nitrogen for several minutes. After having been maintained under stirring at 250° C. for 5 hours, the reaction mass was cooled down to 120° C., stripped under vacuum to remove any trace of unreacted vinyltrimethoxysilane and then cooled down to room temperature. The final liquid product was stored in an air-tight and moisture-free container and then used without any further purification.

### Example 5

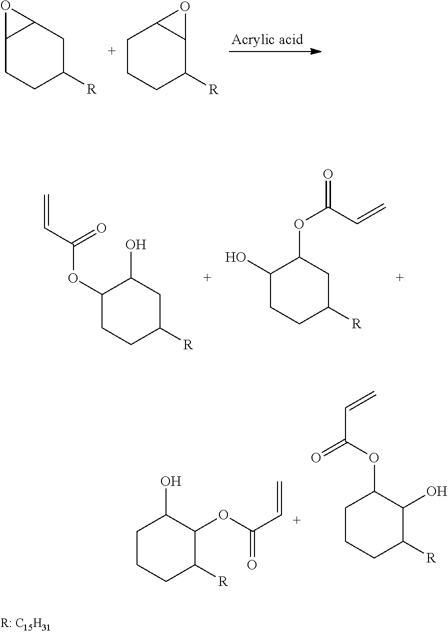

A mixture of pentadecyl-cyclohexenes derived epoxy isomers 2-Pentadecyl-7-oxa-bicyclo[4.1.0]heptane and 3-Pentadecyl-7-oxa-bicyclo[4.1.0]heptane (100 g, EEW 353 g/eq), hydroquinone (0.25 g) and triphenylphosphine (1 g) is stirred a 60° C. for 20 minutes in a round bottom flask equipped with mechanical overhead mixer, thermocouple, condenser and nitrogen inlet.

Acrylic acid (20.4 g) is then added dropwise, keeping temperature around 60° C. At the end of the addition, reaction temperature is increased to 85-90° C. and the system stirred under nitrogen till the epoxy content is below 0.2%. The reaction mass is then cooled down to room temperature and the final product recovered as low viscous liquid than can be used without any further purification.

### Example 6

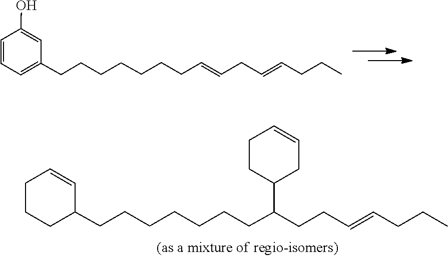

Cardanol (300 g) and phenol (376.4 g) are add in a 1 liter round bottom flask equipped with mechanical overhead mixer, thermocouple, reflux condenser, and heated at 60° C. for 20 minutes under stirring. The pH is then brought to 2.0-2.5 by adding Sulfuric acid 98% (7.1 g) and water (6.8 g) and the reaction mass temperature increased to 80° C. After 8 hours under stirring at this temperature, the product is neutralized with NaOH and stripped under vacuum to remove water and unreacted phenol.

The crude product is then transferred to a Parr reactor, diluted with isopropanol and added with Ni catalyst (3% w/w with respect to substrate). The temperature of the reaction mixture was then raised to 180° C. under hydrogen atmosphere (24 Bar) and maintained for 12 hours. The catalyst was removed via filtration over Celite° and the solvent removed under vacuum.

Phosphoric acid 85% (83.3 g, 1 mol) is then added and the resulting mixture heated to around 90° C. and water is removed under vacuum. Then, while still under vacuum, the mixture is heated to 140-150° C. and the water of reaction is removed until the reaction is complete.

### Industrial Applicability

The major advantage of the present invention is the use of cardanol-derived cyclohexene like regio-isomers, 3-pentadecyl-cyclohexene and 4-pentadecyl-cyclohexene as bio-derived alternative to petro-derived cyclohexene. 3-pentadecyl-cyclohexene and 4-pentadecyl-cyclohexene represent novel versatile tools for the chemical industry, since they can be used in the production of polyesters, polyether, polycarbonates, epoxies, poly-acrylates, poly-methacrylates, amino-alcohols, amines, polyamides, diluents, plasticizers, tougheners and lubricants.

Another advantage of the present invention is the versatility of the methods object of the present invention, that can be applied on cardanol to recover cyclohexene-like monomers and on a variety of cardanol-derivatives with different molecular weight and functionality, thus allowing the synthesis of novel chemical building blocks, still characterized by the presence of pentadecyl-cychlohexene units, along with other functional groups.

The novel structures object of the present invention can impart flexibility even at low temperature, amorphous properties, improved chemical resistance properties and hydrophobicity due to the presence of the —C₁₅H₃₁ group, good weatherability in the final thermoset and thermoplastic polymer matrices, as well as good dilution effect and thermal and oxidative stability.

Cardanol is one of the most promising bio-based material used in the thermoset-industry, by derivatizing through the aromatic ring, the phenolic OH or the side chain double bonds. All the resulting products offer unique features like thermal resistance, high hydrophobicity and chemical resistance, flexibilization effect if the side chain is not modified, low volatility (therefore helping with VOC reduction when low viscosity cardanol-derivatives are used as replacement to potentially dangerous organic solvents).

The present invention provides methods to use cardanol and cardanol-derivatives as starting substrates to recover pentadecyl-cyclohexanol containing products in high purity and yield, followed by their conversion to pentadecyl-cyclohexene like structures (as mixture of isomers, namely 3-pentadecyl-cyclohexene like and 4-pentadecyl-cyclohexene like products). Pentadecyl-cyclohexene derivatives' subsequent derivatization under proper conditions lead to the development of a series of novel cardanol-derivatives, that can be used as such or as novel polymer building blocks. These structures can overcome some of the well-known limitations of cardanol (e.g. UV instability, batch-to-batch consistency) as well as offering novel chemical tools to impart unique chemical and mechanical properties (e.g. low temperature flexibility; low moisture absorption; good weatherability) to both thermosetting and thermoplastic matrices; these structures can be also used in other sectors (e.g. transformer oils, lubricants, fluids for drilling applications), further extending and confirming cardanol applicability in a wide range of applications and the versatility of the novel structures object of the present invention.

The compounds and methods of making the same provided for in the present application can be used in many applications. Examples include, but are not limited to, the use as raw materials for linings, adhesives, alkyds, varnishes, composites, inks, structural polymers, 3D printable polymers, techno-polymers and elastomers, transformer oils, lubricants, antimicrobials, antioxidants, corrosion retardants, cosmetics, detergents, soaps, de-icing products, food, flavors, oil field chemicals, tackifiers, prepolymer chain-extenders, rheology modifiers, electrical and electronic components (potting, castings, encapsulants), personal care products, polymers, engineered plastics, rubbers, sealants, solvents, diluents, toughening agents, plasticizers, surfactants.

In some embodiments, methods for using a compound of formula I, II and III in antimicrobials, antioxidants, adhesives, coatings, corrosion retardants composites, cosmetics, detergents, soaps, de-icing products, elastomers, food, flavors, inks, lubricants, oil field chemicals, personal care products, polymers, structural polymers, engineered plastics, 3D printable polymers, techno-polymers, rubbers, sealants, solvents, surfactants, varnishes etc. are provided.

Certain embodiments and features have been described using a set of numerical upper limits and a set of numerical lower limits. It should be noted that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure are not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A compound of formula I: 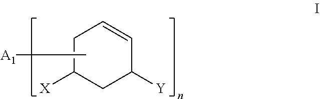
wherein:
A₁ is H; methylene; linear, branched and/or substituted alkyl; linear, branched and/or substituted aryl; linear, branched and/or substituted phenyl; linear, branched and/or substituted alkenyl;
halogen
X and Y are each independently H and R
R is: 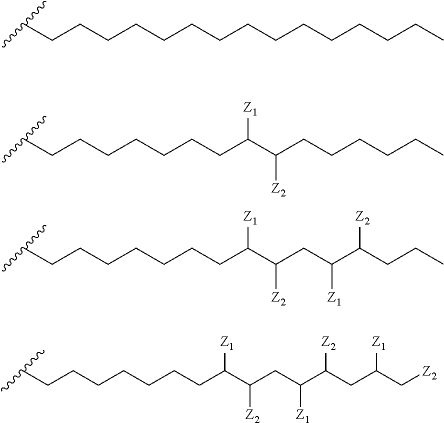
n is independently 0-50
Z₁ is H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
Z₂ is H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.

2. The compound of claim 1, wherein the compound of formula I is a mixture of cyclohexenes: 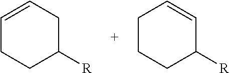
wherein:
R is: 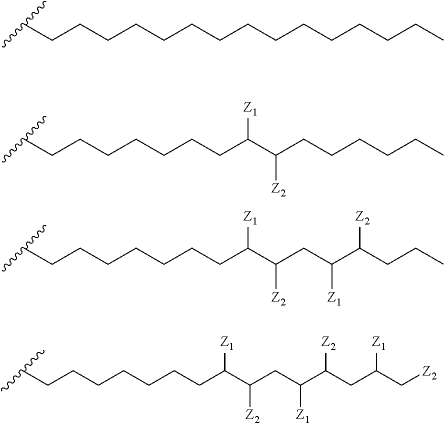
Z₁ is H; halogen; linear, branched and/or substituted alkyl; linear, branched and/or substituted aryl; linear, branched and/or substituted phenyl; linear, branched and/or substituted alkenyl;
triazole, substituted triazole, linear alkoxy, branched alkoxy, —CHCH₃(CH₂)ₘSiR₁R₂R₃
Z₂ is H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.

3. A method for preparing a compound of claims 1 and 2, comprising:
hydrogenating cardanol or a cardanol derivative with hydrogen gas in the presence of at least one catalyst; and an optional solvent as reactants;
heating the reactants at a predetermined temperature and at a predetermined pressure for a predetermined period of time;
removing the catalyst via filtration to produce a fully hydrogenated product;
adding the fully hydrogenated product to a mixture of at least one inorganic compound for a predetermined period of time and under at a predetermined vacuum;
stirring the reaction mixture at a predetermined temperature for a predetermined period of time;
distilling the reaction mixture to recover the compound of Formula I as mixture of isomers

4. The method of claim 3, wherein cardanol has a purity from about 85% to about 99.9%.

5. The method of claim 3, wherein the cardanol derivative has molecular weight from 300 Da to 100000 Da

6. The method of claim 3, wherein the cardanol derivative has a viscosity from 50 cps to 2500000 cps at 25° C.

7. The method of claim 3, wherein the hydrogenation catalyst comprises Pd/C, Pd(OH)₂, Pd/Al₂O₃, Pd/NaY, Ru-PVP NPs, Ru/C, RuCl₃, Ni, and Ni Raney or any combination thereof.

8. The method of claim 3, wherein the inorganic compound comprises phosphoric acid, sulfuric acid, Al₂O₃, Al₂O₃/P₂O₅/pyridine, POCl₃/pyridine or any combination thereof.

9. A compound of formula II: 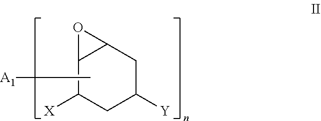
wherein:
A₁ is H; methylene; linear, branched and/or substituted alkyl; linear, branched and/or substituted aryl; linear, branched and/or substituted phenyl; linear, branched and/or substituted alkenyl;
halogen
X and Y are each independently H and R
R is: 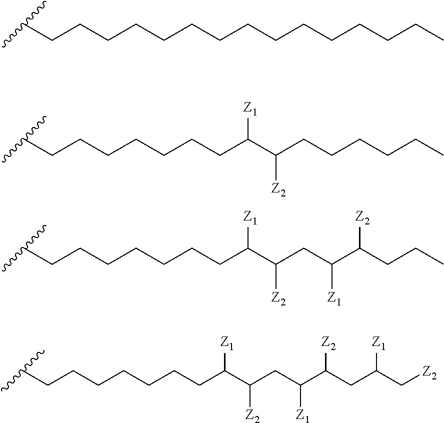
n is independently 0-50
Z₁ is H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
Z₂ is H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like

10. The compound of claim 9, wherein the compound of formula I is a mixture of cyclohexenes: 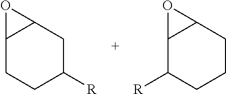
wherein:
R is: 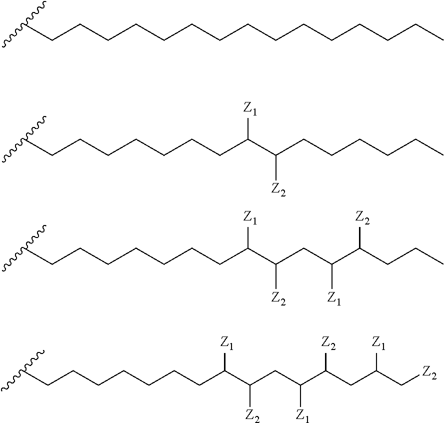
Z₁ is H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
Z₂ is H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.

11. A method for preparing a compound of claims 9 and 10, comprising:
reacting any of the cardanol derivatives of Formula I obtained accordingly to the method described in claim 3 with an oxidant at a predetermined temperature for a predetermined period of time, in presence of an optional solvent;
isolating the resulting epoxy derivatives as mixture of isomers by removal of aqueous phase

12. The method of claim 11, wherein the oxidant comprises a hydrogen peroxide/formic acid mixture, hydrogen peroxide/acetic acid mixture, meta-chloro per-benzoic acid, Oxone or any combination thereof.

13. A compound of formula III: 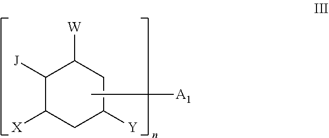
wherein:
A₁ is H; methylene; linear, branched and/or substituted alkyl; linear, branched and/or substituted aryl; linear, branched and/or substituted phenyl; linear, branched and/or substituted alkenyl; halogen
X and Y are each independently H and R
R is: 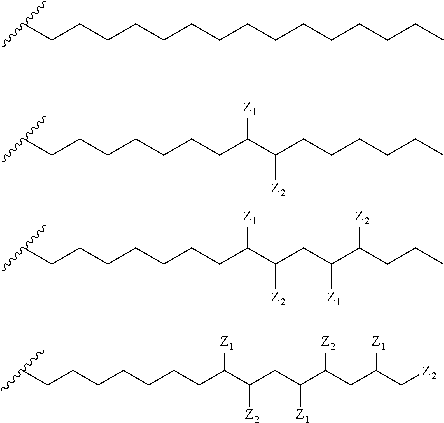
n is independently 0-50
Z₁ is H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
Z₂ is H; halogen; linear, branched and/or substituted alkyl; linear, branched and/or substituted aryl; linear, branched and/or substituted phenyl; linear, branched and/or substituted alkenyl; triazole, substituted triazole, linear alkoxy, branched alkoxy, —CHCH₃(CH₂)ₘSiR₁R₂R₃
m is independently 0-20;
R₁ is independently a hydrolysable group selected from chloro, methoxy, propoxy, methylalkoxy, acetoxy, silazanes, oximes, and the like;
R₂ is independently a saturated or unsaturated hydrocarbon chain of 1-20 carbon atoms, or is hydrolysable group selected from chloro, methoxy, propoxy, methylalkoxy, acetoxy, silazanes, oximes, and the like; and
R₃ is independently a saturated or unsaturated hydrocarbon chain of 1-20 carbon atoms, or is hydrolysable group selected from chloro, methoxy, propoxy, methylalkoxy, acetoxy, silazanes, oximes, and the like.
J and W can be —H, —OH, —OCH₂(CHOCH₂), halogen, —NR₄R₅, —O—R₆, —O—CO—R₇, —O—CO—OR₈;
wherein R₄, R₅, R₆, R₇, R₈ are independently H, —CH₃, alkyl, vinyl, alkenyl, alkoxy, cycloalkyl, aryl, heteroaryl, and —O(CH₂CHR₉O)_{q}CH₂CHR₉OH; and
R₉ is H or CH₃
q is 1-100.
